(19) United States
(12) Patent Application Publication
(10) Pub. No.: US 2010/0010582 A1
(43) Pub. Date: Jan. 14, 2010

(54) MEDICAL SYSTEM AND METHOD FOR SETTING PROGRAMMABLE HEAT LIMITS

(75) Inventors: Rafael Carbunaru, Valley Village, CA (US); Que T. Doan, West Hills, CA (US)

Correspondence Address: VISTA IP LAW GROUP LLP/BSC - NEUROMODULATION, 2040 MAIN STREET , 9TH FLOOR, IRVINE, CA, 92614, US

(73) Assignee: BOSTON SCIENTIFIC NEUROMODULATION CORPORATION, Valencia, CA (US)

(21) Appl. No.: 12/501,226

(22) Filed: Jul. 10, 2009

Related U.S. Application Data
(60) Provisional application No. 61/080,160, filed on Jul. 11, 2008.

(51) Int. Cl. A61N 1/378

(52) U.S. Cl. 607/61

(57) ABSTRACT

An external charger transmits energy to charge an implanted medical device. A sensor measures a parameter that is correlated to a temperature that is adjacent to the external charger. This parameter is indicative of the temperature or an amount of heat that is generated during the charging of the implanted medical device by the external charger. The temperature is compared to a user programmable temperature threshold and based on the comparison, the charge rate or output power of the external charger or input power of the implanted medical device is adjusted to reduce the heat generated by the charging. The user programmable temperature threshold is set to an optimum charge rate whereby the temperature that is generated during charging of the implanted medical device by the external charger feels comfortable to the user.

### RELATED APPLICATION

The present application claims the benefit under 35 U.S.C. §119 to U.S. provisional patent application Ser. No. 61/080,160, filed Jul. 11, 2008. The foregoing application is hereby incorporated by reference into the present application in its entirety.

### FIELD OF THE INVENTION

The present invention relates to implantable devices, and more particularly, to devices for transcutaneously recharging devices implanted within patients.

### BACKGROUND OF THE INVENTION

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder sublaxation, etc. The present invention may find applicability in all such applications, although the description that follows will generally focus on the use of the invention within a spinal cord stimulation system, such as that disclosed in U.S. Pat. No. 6,516,227, which is expressly incorporated herein.

Spinal cord stimulation is a well-accepted clinical method for reducing pain in certain populations of patients. A spinal cord stimulation (SCS) system typically includes an implantable pulse generator and at least stimulation electrode lead that carries electrodes that are arranged in a desired pattern and spacing to create an electrode array. Individual wires within the electrode lead(s) connect with each electrode in the array. The electrode lead(s) is typically implanted along the dura of the spinal cord, with the electrode lead(s) exiting the spinal column, where it can generally be coupled to one or more electrode lead extensions. The electrode lead extension(s), in turn, are typically tunneled around the torso of the patient to a subcutaneous pocket where the implantable pulse generator is implanted. Alternatively, the electrode(s) lead may be directly coupled to the implantable pulse generator. For examples of other SCS systems and other stimulation systems, see U.S. Pat. Nos. 3,646,940 and 3,822,708, which are hereby incorporated by reference in their entireties.

Of course, implantable pulse generators are active devices requiring energy for operation. Oftentimes, it is desirable to recharge an implanted pulse generator via an external charger, so that a surgical procedure to replace a power depleted implantable pulse generator can be avoided. To wirelessly convey energy between the external charger and the implanted pulse generator, the recharger typically includes an alternating current (AC) charging coil that supplies energy to a similar charging coil located in or on the implantable pulse generator. The energy received by the charging coil located on the implantable pulse generator can then be used to directly power the electronic componentry contained within the pulse generator, or can be stored in a rechargeable battery within the pulse generator, which can then be used to power the electronic componentry on-demand.

To provide efficient power transmission through tissue from the external charger to the implanted pulse generator, it is paramount that the charging coil located in or on the implantable pulse generator be spatially arranged relative to the corresponding AC coil of the external charger in a suitable manner. That is, efficient power transmission through the patient's skin from the external charger to the implantable pulse generator via inductive coupling requires constant close alignment between the two devices. To ensure that such constant close alignment is achieved, the external charger is typically placed against the skin of the patient, thereby maintaining or optimizing the rate at which the implantable pulse generator is charged.

During its normal operation, the external charger necessarily generates heat that could be intolerable and unsafe if left unregulated. To address the generation of heat, external chargers typically include pre-programmed or pre-set maximum temperature safety limits, so that a patient is not harmed when the external charger is placed against the patient's skin during charging of the implantable pulse generator. While generally acceptable safety limits have been regulated for the population as a whole, those safety limits may still lead to discomfort of the patient, since heat sensitivity varies from one patient to another. For example, the heat may be uncomfortable due to the age of the patient, or some other medical condition which makes a patient more sensitive to heat. As another example, the patient may feel excessive heat when the external charger is operated at standard or normal conditions, but is left in the same or general area for prolonged period of time during charging.

Thus, a patient could still feel discomfort if the safety limit is set too high. On the other hand, if the safety limit is set too low, charging of the IPG may occur too slowly. As a result, some patients may not benefit from the entire potential of the IPG use.

Therefore, there is a need for an improved system that regulates the heat generated by an external charger.

### SUMMARY OF THE INVENTION

In accordance with a first aspect of the present inventions, a medical system is provided. The medical system comprises an implantable medical device (e.g., a neurostimulation device, such as an implantable pulse generator), and an external device configured for transcutaneously coupling energy into the implantable medical device. In one embodiment, the external device is an external charger, in which case, the coupled energy charges the implantable medical device. The medical system further comprises a sensor configured for measuring a parameter correlated to a temperature generated by the external device during coupling of the energy into the implantable medical device. The measured parameter can be, e.g., the temperature, itself, or one of an input power of the implantable medical device and an output power of the external device. In one embodiment, the temperature that is measured is the temperature in the external device. The temperature may be, e.g., an instantaneous temperature or an average temperature.

The medical system further comprises memory configured for storing a user programmable threshold, and a processor configured for comparing the measured parameter to the user programmable threshold, and for controlling the temperature based on the comparison (e.g., by adjusting a charge rate of the implantable medical device or alternately terminating and initiating the transcutaneous coupling of energy into the implantable medical device. For example, the processor may be configured for In one embodiment, the sensor and the processor are contained in the implantable medical device. In another embodiment, the sensor and processor are contained in the external device. In still another embodiment, the sensor is contained in one of the implantable medical device, and the processor is contained in another of the implantable medical device and the external device. In this case, the system further comprises a communications device configured for communicating the measured parameter from the one of the implantable device and the external device to the other of the implantable medical device and the external device. In an optional embodiment, the system may further comprise an external programmer configured for programming the user programmable threshold in the memory. In this case, the processor may be contained in the external programmer.

In accordance with a second aspect of the present inventions, an external device for providing energy to an implantable medical device is provided. The external device comprises an alternating current (AC) coil configured for transcutaneously conveying the energy to the implantable medical device, and a sensor configured for measuring a parameter correlated to a temperature generated by the external device during the transcutaneous conveyance of the energy to the implantable medical device. The external device further comprises memory configured for storing a user programmable threshold, and a processor configured for comparing the measured parameter to the user programmable threshold, and for controlling the temperature based on the comparison. The measured parameter and manner in which the temperature generated by the external device can be same as those described above. The external device further comprises a housing (e.g., a hand-held housing) containing the AC coil, sensor, memory, and processor. In an optional embodiment, the external device further comprises a source of electrical power configured for providing the energy to the AC coil.

In accordance with a third aspect of the present inventions, a method for regulating a temperature generated by an external device is provided. The method comprises transcutaneously coupling energy from the external device to a medical device (e.g., a neurostimulation device, such as an implantable pulse generator) implanted within a patient. In one method, the external device is an external charger, in which case, the transcutaneous coupling of the energy charges the external charger. The method comprises measuring a parameter correlated to the temperature during the transcutaneous coupling of the energy to the medical device. The method further comprises modifying a stored threshold, e.g., by the patient. One method further comprises determining a temperature that is comfortable to the patient, wherein the stored threshold is modified based on the determined comfortable temperature. The method further comprises comparing the measured parameter to the stored threshold, and controlling the temperature based on the comparison. The measured parameter and manner in which the temperature generated by the external device can be same as those described above.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is plan view of one embodiment of a spinal cord stimulation (SCS) system arranged in accordance with the present inventions;

FIG. 2 is a plan view of the SCS system of FIG. 1 in use with a patient;

FIG. 3 is a perspective view of one embodiment of an external charger used in the SCS system of FIG. 1;

FIG. 4 is a block diagram of the internal components of one embodiment of an external charger, sensor and implantable pulse generator used in the SCS system of FIG. 1; and

FIG. 5 is a block diagram of the internal components of another embodiment of an external charger, sensor and implantable pulse generator used in the SCS system of FIG. 1.

### DETAILED DESCRIPTION OF THE EMBODIMENTS

At the outset, it is noted that the present invention may be used with an implantable pulse generator (IPG) or any other similar electrical stimulator, which may be used as a component of numerous different types of stimulation systems. The description that follows relates to a spinal cord stimulation (SCS) system. While the invention lends itself well to SCS applications, the invention in its broadest aspects may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical and deep brain stimulator, peripheral nerve stimulator, or in any other neural stimulator configured to treat urinary incontinence, sleep apnea, shoulder sublaxation, etc.

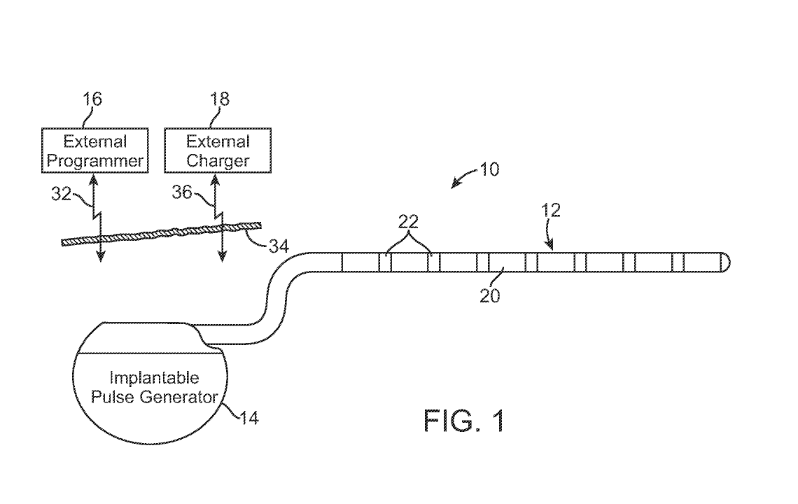

Turning first to FIG. 1, a preferred SCS system 10 generally comprises an implantable neurostimulation lead 12, an implantable pulse generator (IPG) 14, an external (non-implanted) programmer 16, and an external (non-implanted) charger 18. In the illustrated embodiment, the lead 12 is a percutaneous lead and, to that end, includes a plurality of in-line electrodes 22 carried on a flexible body 20. Alternatively, the lead 12 may take the form of a paddle lead. The IPG 14 is electrically coupled to the lead 12 in order to direct electrical stimulation energy to each of the electrodes 22. The IPG 14 includes an outer case formed from an electrically conductive, biocompatible material, such as titanium and, in some instances, will function as an electrode. The case forms a hermetically sealed compartment wherein the electronic and other components are protected from the body tissue and fluids. For purposes of brevity, the electronic components of the IPG 14, with the exception of the components needed to facilitate the recharging function (described below), will not be described herein. Details of the IPG 14, including the battery, antenna coil, and telemetry and charging circuitry, are disclosed in U.S. Pat. No. 6,516,227, which is expressly incorporated herein by reference.

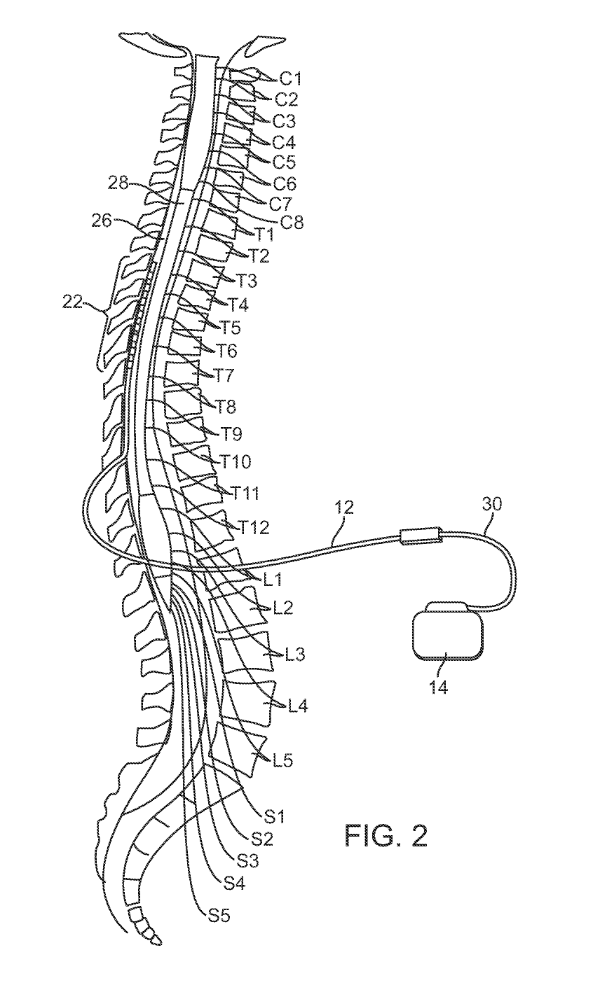

As shown in FIG. 2, the neurostimulation lead 12 is implanted within the epidural space 26 of a patient through the use of a percutaneous needle or other convention technique, so as to be in close proximity to the spinal cord 28. Once in place, the electrodes 22 may be used to supply stimulation energy to the spinal cord 28 or nerve roots. The preferred placement of the lead 12 is such, that the electrodes 22 are adjacent, i.e., resting upon, the nerve area to be stimulated. Due to the lack of space near the location where the lead 12 exits the epidural space 26, the IPG 14 is generally implanted in a surgically-made pocket either in the abdomen or above the buttocks. The IPG 14 may, of course, also be implanted in other locations of the patient's body. A lead extension 30 may facilitate locating the IPG 14 away from the exit point of the lead 12.

Referring back to FIG. 1, the IPG 14 is programmed, or controlled, through the use of the external programmer 16. The external programmer 16 is transcutaneously coupled to the IPG 14 through a suitable communications link (represented by the arrow 32) that passes through the patient's skin 34. Suitable links include, but are not limited to radio frequency (RF) links, inductive links, optical links, and magnetic links. For purposes of brevity, the electronic components of the external programmer 16 will not be described herein. Details of the external programmer 16, including the control circuitry, processing circuitry, and telemetry circuitry, are disclosed in U.S. Pat. No. 6,516,227, which has been previously incorporated herein by reference.

The external charger 18 is transcutaneously coupled to the IPG 14 through a suitable link (represented by the arrow 36) that passes through the patient's skin 34, thereby coupling power into the IPG 14 for the purpose of operating the IPG 14 or replenishing a power source, such as a rechargeable battery (e.g., a Lithium Ion battery), within the IPG 14. In the illustrated embodiment, the link 36 is an inductive link; that is, energy from the external charger 18 is coupled to the battery within the IPG 14 via electromagnetic coupling. Once power is induced in the charging coil in the IPG 14, charge control circuitry within the IPG 14 provides the power charging protocol to charge the battery. As will be described in further detail below, the external charger 18 generates an audible tone when misaligned with the IPG 14 to alert the user to adjust the positioning of the external charger 18 relative to the IPG 14. The external charger 18 is designed to charge the battery of the IPG 14 to 80% capacity in two hours, and to 100% in three hours, at implant depths of up to 2.5 cm. When charging is complete, the external charger 18 generates an audible tone to alert the user to decouple the external charger 18 from the IPG 14.

The charger 18 can charge the implantable medical device 14 using a constant or varying power or charge rate. Instead of having the charge rate or output power being held at a constant rate (e.g., the optimum or maximum charge rate), the charge rate or output power can be varied or changed over a period of time. For example, the charge rate could be set to the optimum charge rate for a period of time, then lowered for another period of time, and repeating these steps until the IPG 14 is fully charged. In another example, the charge rate could be set to the optimum charge rate for a period of time, and then gradually lowered over another period of time.

Once the IPG 14 has been programmed, and its power source has been charged or otherwise replenished, the IPG 14 may function as programmed without the external programmer 16 being present. While the external programmer 16 and external charger 18 are described herein as two separate and distinct units, it should be appreciated that the functionality of the external programmer 16 and external charger 18 can be combined into a single unit. It should be noted that rather than an IPG, the SCS system 10 may alternatively utilize an implantable receiver-stimulator (not shown) connected to leads 12, 14. In this case, the power source, e.g., a battery, for powering the implanted receiver, as well as control circuitry to command the receiver-stimulator, will be contained in an external controller inductively coupled to the receiver-stimulator via an electromagnetic link.

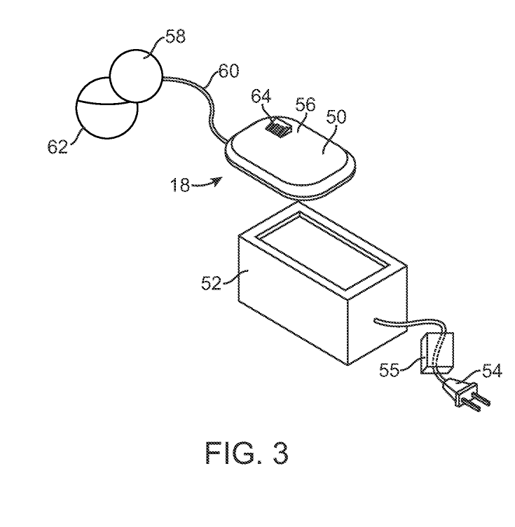

Referring now to FIG. 3, the external components of the external charger 18 will be described. In this embodiment, the external charger 18 takes the form of a two-part system comprising a portable charger 50 and a charging base station 52. The charging base station 52 includes an AC plug 54, so that it can be easily plugged into any standard 110 volt alternating current (VAC) or 200 VAC outlet. The charging base station 52 further includes an AC/DC transformer 55, which provides a suitable DC voltage (e.g., 5 VDC) to the circuitry within the charging base station 52.

The portable charger 50 includes a housing 56 for containing circuitry, and in particular, the recharging circuitry and battery (not shown in FIG. 3), which will be discussed in further detail below. The housing 56 is shaped and designed in a manner that allows the portable charger 50 to be detachably inserted into the charging base station 52, thereby allowing the portable charger 50, itself, to be recharged. Thus, both the IPG 14 and the portable charger 50 are rechargeable. The portable charger 50 may be returned to the charging base station 52 between uses.

In the illustrated embodiment, the portable charger 50 includes a charging head 58 connected to the housing 56 by way of a suitable flexible cable 60. The charging head 58 houses the AC coil (not shown in FIG. 3) from which the charging energy is transmitted. The portable charger 50 further includes a disposable adhesive pouch 62 or Velcro® strip or patch, which may be placed on the patient's skin over the location where the IPG 14 is implanted. Thus, the charging head 58 may be simply slid into the pouch 62, or fastened to the strip or patch, so that it can be located in proximity to the IPG 14 (e.g., 2-3 cm). In an alternative embodiment, the portable charger 50 does not include a separate charging head, but instead includes a single housing that contains the recharging circuitry, a sensor, the battery, and the AC coil. The portable charger 50 includes a bar charge indicator 64 located on the housing 56, which provides a visual indication of the strength of the charging between the charging head 58 and IPG 14 in the form of bars.

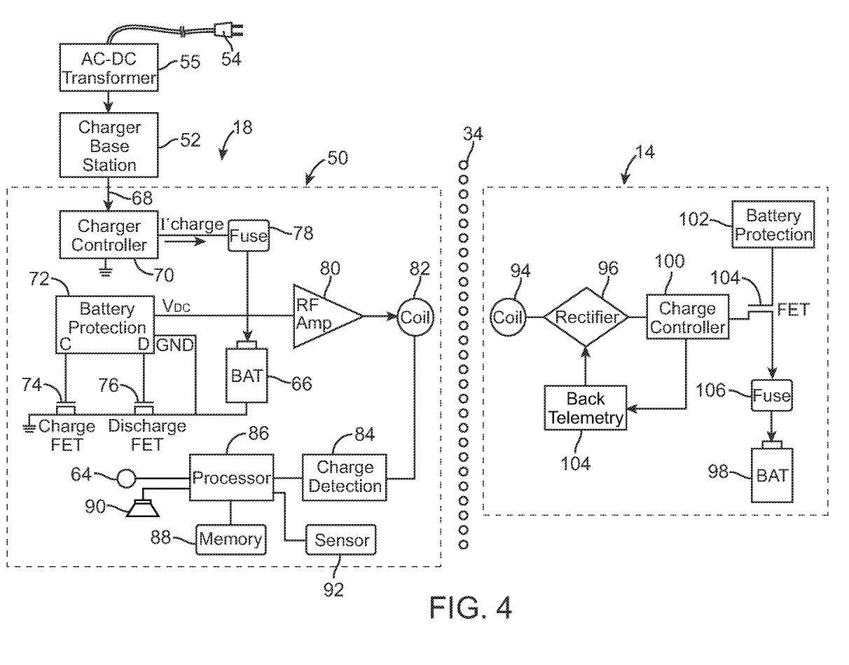

Referring to FIG. 4, the recharging elements of the IPG 14 and portable charger 50 will now be described. It should be noted that the diagram of FIG. 4 is functional only, and is not intended to be limiting. Those of skill in the art, given the descriptions presented herein, should be able to readily fashion numerous types of recharging circuits, or equivalent circuits, that carry out the functions indicated and described.

As previously discussed above, the external charger 18 and IPG 14 are shown inductively coupled together through the patient's skin 34 (shown by dotted line) via the inductive link 36 (shown by wavy arrow). The portable charger 50 includes a battery 66, which in the illustrated embodiment is a rechargeable battery, such as a Lithium Ion battery. Thus, when a recharge is needed, energy (shown by arrow 68) is coupled to the battery 66 via the charging base station 52 in a conventional manner. In the illustrated embodiment, the battery 66 is fully charged in approximately four hours. Once the battery 66 is fully charged, it has enough energy to fully recharge the battery of the IPG 14. If the portable charger 50 is not used and left on charger base station 52, the battery 66 will self-discharge at a rate of about 10% per month. Alternatively, the battery 66 may be a replaceable battery.

The portable charger 50 includes a charge controller 70, which serves to convert the DC power from the AC/DC transformer 55 to the proper charge current and voltage for the battery 66, a battery protection circuit 72, which monitors the voltage and current of the battery 66 to ensure safe operation via operation of FET switches 74, 76, and a fuse 78 that disconnects the battery 66 in response to an excessive current condition that occurs over an extended period of time. Further details discussing this control and protection circuitry are described in U.S. Pat. No. 6,516,227, which has been previously incorporated herein by reference.

The portable charger 50 further includes a power amplifier 80, and in particular a radio frequency (RF) amplifier, for converting the DC power from the battery 66 to a large alternating current. The power amplifier may take the form of an E-class amplifier. The portable charger 50 further includes an antenna 82, and in particular a coil, configured for transmitting the alternating current to the IPG 14 via inductive coupling. The coil 82 may comprise a 36 turn, single layer, 30 AWG copper air-core coil having a typical inductance of 45 μH and a DC resistance of about 1.15 Ω. The coil 82 may be tuned for a resonance at 80 KHz with a parallel capacitor (not shown).

The portable charger 50 comprises charge detection circuitry 84 for detecting an electrical parameter indicative of the charge rate of the IPG 14, and a processor 86 for determining the charging qualities of the IPG 14, and in particular, when the IPG 14 is fully charged and when the portable charger 50 is aligned/misaligned with the IPG 14, based on the detected electrical parameter. The portable charger 50 further comprises memory 88 for storing an electrical parameter threshold value that the processor 86 uses to determine misalignment between the portable charger 50 and IPG 14. The memory 88 also stores a computer program used by the processor 86 to perform the functions described below. The portable charger 50 also includes an indicator 90 in the form of an audio transducer (speaker), which signals the user with an audible tone when the battery 98 of the IPG 14 is fully charged and when the portable charger 50 is misaligned with the IPG 14.

The IPG 14 includes an antenna 94, and in particular a coil, configured for receiving the alternating current from the portable charger 50 via the inductive coupling. The coil 94 may be identical to, and preferably has the same resonant frequency as, the coil 82 of the portable charger 50. The IPG 14 further comprises rectifier circuitry 96 for converting the alternating current back to DC power. The rectifier circuitry 96 may, e.g., take the form of a bridge rectifier circuit. The IPG 14 further includes a rechargeable battery 98, such as a Lithium Ion battery, which is charged by the DC power output by the rectifier circuitry 96. In the illustrated embodiment, the battery 98 can be fully charged by the portable charger 50 in under three hours (80% charge in two hours).

The IPG 14 includes a charge controller 100, which serves to convert the DC power from the rectifier circuitry 96 to the proper charge current and voltage for the battery 98, a battery protection circuit 102, which monitors the voltage and current of the battery 98 to ensure safe operation via operation of a FET switch 104, and a fuse 96 that disconnects the battery 98 in response to an excessive current condition that occurs over an extended period of time. Further details discussing this control and protection circuitry are described in U.S. Pat. No. 6,516,227, which has been previously incorporated herein by reference.

Significantly, the charger 50 is capable of regulating the temperature generated by it, and in particular coil 82, during the charging of the IPG 14 in order to avoid injuring the patient. Preferably, the temperature is adjacent the IPG 14, and more preferably, on the side of the charger 50 (i.e., the side on which the coil 82 is located) that is intended to be placed against the skin of the patient. To this end, the charger 50 further comprises a temperature sensor 92 configured for measuring the temperature generated by the charger 50 during the charging of the IPG 14.

The memory 88 stores a user programmable threshold, and in the illustrated embodiment, a user programmable temperature threshold. The temperature threshold may be programmed by the user, e.g., using the external programmer 16 (shown in FIG. 1). In this case, the external programmer 16 wirelessly transmits the temperature threshold information to the charger 50, which would include an antenna (not shown) for receiving the temperature threshold information. The processor 86 would then acquire the temperature threshold information from the antenna and store it in the memory 88. Alternatively, the external programmer 16, itself, may include a programming device, such as a dial, that can be manipulated by the user to program the temperature threshold into the memory 88.

The processor 86 is configured for comparing the measured temperature to the user programmable temperature threshold and controlling the temperature based on the comparison. In one embodiment, the temperature may be controlled by controlling the RF amplifier 80 to adjust the charge rate of the IPG 14 or alternately terminating and initiating the charging of the IPG 14. For example, if the measured temperature exceeds the temperature threshold (i.e., an excessive temperature is detected), the processor 86 may decrease the charge rate of the IPG 14 or temporarily terminating charging of the IPG 14, thereby decreasing the temperature generated by the IPG 14 to a level that falls within an acceptable level for the user. If the measured temperature does not exceed a temperature threshold (i.e., an excessive temperature is detected), the processor 86 may continue the charging operation without interruption. Once the measured temperature drops below a temperature threshold, the processor 86 may increase the charge rate of the IPG 14 or reinitiate charging of the IPG 14. Preferably, hysteresis may be built into the IPG 14, such that the temperature threshold that triggers increasing the charge rate or initiating charging may be a certain level below the temperature threshold that triggers decreasing the charge rate or terminating charging, thereby maintaining stability of the charging control.

The user programmable temperature threshold can be set to an optimum level at which the portable charger 50 is generating an acceptable amount of heat for a particular patient. For example, in one embodiment, the user programmable temperature threshold value can simply be manually programmed by a user. The user performs a series of tests to determine the optimum charge rate or output power of the external charger. The tests could involve setting different charge rates of the portable charger 50, and determining whether the resulting temperature is acceptable for the patient. The tests would naturally stay within the prescribed safety limits. The tests would not cause the patient any harm from excessive heat, especially one where a patient would be burned. The tests can be performed by the patient, but it is recommended that a doctor, a nurse, a medical clinician or other medical professional perform the tests to determine the optimum charge rate.

The optimum charge rate will be one in which an acceptable level of heat is generated by charger 50 when charging the IPG 14. The faster the charge rate, the less time it takes to charge the IPG 14. Once the maximum temperature is found that is acceptable to a patient for the highest charge rate within the prescribed safety limits, the user programmable temperature threshold is set to this value and is stored in memory 88 (e.g., via operation of the external programmer 16 or the charger 50 as described above).

In an alternative embodiment, instead of setting the user programmable temperature threshold to an instantaneous temperature (e.g., the maximum or optimum temperature), the threshold could be set to an average temperature. This accounts for the situation where the charger 50 is charging the IPG 14 for a prolonged or extended period of time. When the charger 50 is charging the IPG 14, a user may not feel discomfort for smaller durations of time, for example, 10 minutes. However, if the charger 50 is charging the IPG 14 over a prolonged period of time, for example, 30 minutes, a user could experience discomfort due to the period of time that the charger 50 is taking to charge the IPG 14. So in this embodiment, the processor 86 controls the average temperature that is generated by the charger 50 over a period of time, so that the patient will not experience any heat-related discomfort when the IPG 14 is being charged over a longer period of time.

Although the charger 50 has been described as measuring the temperature, itself, the charger 50 may be configured for measuring a different parameter that can be correlated to the temperature generated by the charger 50 during the charging of the IPG 14. For example, the measured parameter can be an output power of the charger 50, which can be sensed by the charge detection circuitry 84. The output charge power is directly proportional to the temperature generated by the charger 50 (i.e., the higher the output power the higher the temperature generated by the charger 50, and the lower the output power the lower the temperature generated by the charger 50).

In this case, the user programmable threshold may take the form of an output charging power threshold. Alternatively, if the user programming threshold may still be a temperature, in which case, the measured output power would need to be correlated to an estimated temperature that would be compared to the user programmable threshold temperature. There are different approaches to correlating a parameter to a temperature measurement. One approach would be to perform some type of computation to determine the temperature. Another approach would be to search and find the corresponding temperature in an empirical table (that could be stored in memory 88) based on the value of the parameter.

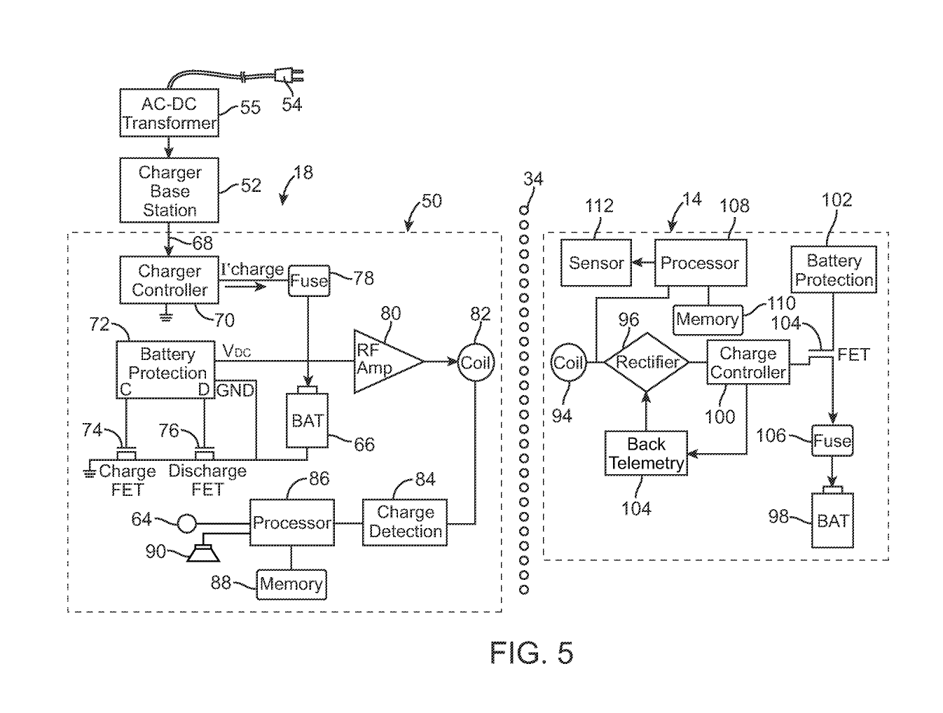

While the processor and sensor that performs the temperature regulation function has been described as being contained in the external charger 50, as shown in FIG. 4, it should be appreciated that the processor and sensor can be contained in the IPG. In particular, as shown in FIG. 5, the IPG 14 further includes a processor 108, memory 110, and a sensor 112 that, with respect to the temperature regulation function, operate in the same manner as the processor 86, memory 88, and sensor 92 described above, with the memory 110 storing the user programmable temperature threshold. However, instead of measuring the temperature within the charger 50, the sensor 112 measures the input charging power at the coil 94.

Because the input charging power is related to the output charging power at the coil 82, and thus, the temperature within the charger 50, the processor 108 can derive an estimated temperature from the measured input charging power in a similar manner described above with respect to the derivation of the estimated temperature from the measured output charging power (e.g., computationally or empirically). The processor 108 can then compare the estimated temperature to the user programmable temperature threshold. In the case where a different threshold, such as an input charging power threshold is used, the measured input charging power can be directly compared to the input charging power threshold. The processor 108 may indirectly regulate the temperature generated by the charger 50 by modifying the charge rate of the IPG 14, for example, by adjusting the electrical impedance of the coil 94.

Alternatively, the commands can be generated by the processor 108 and transmitted from the IPG 14 to the charger 50 using the coils 82, 94. For example, the back telemetry circuit 104 may modulate information onto the secondary load of the IPG 14, which will alter the reflected impedance into the coil 82 of the charger 50 for detection by the charge detection circuitry 83. Alternatively, the commands can be transmitted from the IPG 14 to the charger 50 using a conventional RF transceiver and antenna system. In either event, the commands, once received by the charger 50, can then be interpreted by the processor 86 and used to regulate the temperature generated by the charger 50 in the same manner described above.

In still another embodiment, the processor that performs the temperature regulation function can be contained in the IPG 14 or the external programmer 16. For example, the sensor 92 within the charger 50 can measure the temperature, which information can then be transmitted to the processor in the IPG 14 or external programmer 16. The processor can compare the measured temperature to a user programmable temperature threshold stored within memory associated with the IPG 16 or external programmer 16, and indirectly control the temperature generated by the charger 50 by generating and transmitting commands to the charger 50. The processor 86 in the charger 50 can then use the commands to adjust the charge rate of the IPG or alternately initiate and terminate charging of the IPG 14.

While the present inventions lend themselves to regulating the temperature within an external device, such as an external charger, or should be appreciated that the temperature within an implanted device may be regulated in the same manner; that is, by using a user programmable threshold, sensing a parameter correlated to the temperature generated by the implanted device, comparing the measured parameter to the user programmable threshold, and controlling the temperature generated by the implanted device based on the comparison.

Although particular embodiments of the present inventions have been shown and described, it will be understood that it is not intended to limit the present inventions to the preferred embodiments. It will be obvious to those skilled in the art that various changes and modifications may be made without departing from the spirit and scope of the present inventions. Thus, the present inventions are intended to cover alternatives, modifications and equivalents, which may be included within the spirit and scope of the present inventions as defined herein.

What is claimed is:

1. A medical system, comprising:
an implantable medical device;
an external device configured for transcutaneously coupling energy into the implantable medical device;
a sensor configured for measuring a parameter correlated to a temperature generated by the external device during coupling of the energy into the implantable medical device;
memory configured for storing a user programmable threshold; and
a processor configured for comparing the measured parameter to the user programmable threshold, and for controlling the temperature based on the comparison.

2. The system of claim 1, wherein the implantable medical device is a neurostimulation device.

3. The system of claim 1, wherein the implantable medical device is an implantable pulse generator.

4. The system of claim 1, wherein the external device is an external charger.

5. The system of claim 1, wherein the measured parameter is the temperature.

6. The system of claim 1, wherein the measured parameter is one of an input power of the implantable medical device and an output power of the external device.

7. The system of claim 1, wherein the temperature is internal to the external device.

8. The system of claim 1, wherein the sensor and the processor are contained in the implantable medical device.

9. The system of claim 1, wherein the sensor and processor are contained in the external device.

10. The system of claim 1, wherein the sensor is contained in one of the implantable medical device, and the processor is contained in another of the implantable medical device and the external device, the system further comprising a communications device configured for communicating the measured parameter from the one of the implantable device and the external device to the other of the implantable medical device and the external device.

11. The system of claim 1, wherein the processor is configured for controlling the temperature by adjusting a charge rate of the implantable medical device.

12. The system of claim 1, wherein the processor is configured for controlling the temperature by alternately terminating and initiating the transcutaneous coupling of energy into the implantable medical device.

13. The system of claim 1, wherein the temperature is an instantaneous temperature.

14. The system of claim 1, wherein the temperature is an average temperature.

15. The system of claim 1, further comprising an external programmer configured for programming the user programmable threshold in the memory.

16. The system of claim 15, wherein the processor is contained within the external programmer.

17. An external device for providing energy to an implantable medical device, comprising:
an alternating current (AC) coil configured for transcutaneously conveying the energy to the implantable medical device;
a sensor configured for measuring a parameter correlated to a temperature generated by the external device during the transcutaneous conveyance of the energy to the implantable medical device;
memory configured for storing a user programmable threshold;
a processor configured for comparing the measured parameter to the user programmable threshold, and for controlling the temperature based on the comparison; and
a housing containing the AC coil, sensor, memory, and processor.

18. The external device of claim 17, wherein the measured parameter is the temperature.

19. The external device of claim 17, wherein the measured parameter is an output charging power of the external device.

20. The external device of claim 17, wherein the temperature is internal to the external device.

21. The external device of claim 17, wherein the processor is configured for controlling the temperature by adjusting a charge rate of the implantable medical device.

22. The external device of claim 17, wherein the processor is configured for controlling the temperature by alternately terminating and initiating the transcutaneous coupling of energy into the implantable medical device.

23. The external device of claim 17, wherein the temperature is an instantaneous temperature.

24. The external device of claim 17, wherein the temperature is an average temperature.

25. The external device of claim 17, wherein the housing is a hand-held housing.

26. The external device of claim 17, further comprising a source of electrical power configured for providing the energy to the AC coil.

27. A method for regulating a temperature generated by an external device, comprising:
transcutaneously coupling energy from the external device to a medical device implanted within a patient;
measuring a parameter correlated to the temperature during the transcutaneous coupling of the energy to the medical device;
modifying a stored threshold;
comparing the measured parameter to the stored threshold; and
controlling the temperature based on the comparison.

28. The method of claim 27, wherein the medical device is a neurostimulation device.

29. The method of claim 27, wherein the medical device is an implantable pulse generator.

30. The method of claim 27, wherein the external device is an external charger, and the transcutaneous coupling of the energy charges the external charger.

31. The method of claim 27, wherein the measured parameter is the temperature.

32. The method of claim 27, wherein the measured parameter is one of an input charging power of the medical device and an output charging power of the external device.

33. The method of claim 27, wherein the temperature is internal to the external device.

34. The method of claim 27, wherein the temperature is controlled by adjusting a charge rate of the medical device.

35. The method of claim 27, wherein the temperature is controlled by alternately terminating and initiating the transcutaneous coupling of energy into the implantable medical device.

36. The method of claim 27, wherein the temperature is an instantaneous temperature.

37. The method of claim 27, wherein the temperature is an average temperature.

38. The method of claim 27, wherein the threshold is modified by the patient.

39. The method of claim 27, further comprising determining a temperature that is comfortable to the patient, wherein the stored threshold is modified based on the determined comfortable temperature.
